(19) United States
(12) Patent Application Publication
(10) Pub. No.: US 2008/0053433 A1
(43) Pub. Date: Mar. 6, 2008

(54) K.P. Buteyko Method Of Treating Hypocarbia Diseases

(76) Inventors: Lyudmila Dmitrievna Buteyko, Moscow (RU); Andrey Evgenievich Novozhilov, Moscow (RU)

Correspondence Address: PAULEY PETERSEN & ERICKSON, 2800 WEST HIGGINS ROAD, SUITE 365, HOFFMAN ESTATES, IL, 60195, US

(21) Appl. No.: 11/587,915

(22) PCT Filed: Apr. 18, 2005
(86) PCT No.: PCT/RU05/00200

§ 371 Date: Oct. 26, 2006

(30) Foreign Application Priority Data
Apr. 29, 2004 (RU) 2004113277

(51) Int. Cl. A61M 16/00

(52) U.S. Cl. 128/200.24

(57) ABSTRACT

In the field of medicine, a method for treating hypocarbia by decreasing lung ventilation while monitoring the CO₂ content in the exhaled air. K. P. Buteyko method of treating hypocarbia diseases and states includes decreasing lung ventilation to the extent tolerant for a patient at rest and during exercise. An increase rate of CO₂ in the exhaled air is not more than 2 mm Hg per day, at rest, 11 mm Hg during exercise, up to a level of 32.1 mm Hg in the case of eliminating diseases and symptoms, and up to 55 mm Hg in the case of long-term clinical remission.

### BACKGROUND OF THE INVENTION

1. Field of the Invention

This invention relates to the field of medicine and is intended for treating hypocarbia by decreasing lung ventilation, to the extent tolerant for patient at rest and during exercise, while monitoring the CO₂ content in the exhaled air.

2. Discussion of Related Art

From the prior art, a method of treating hemohypocarbia in bronchial asthma is known, according to a certificate of authorship No. 1067640, A61M 16/00, which relates to decreasing lung ventilation to the extent tolerant for a patient, monitoring the carbon dioxide level in the exhaled air and maintaining its increase rate at not more than 3-7 mm Hg per day, in order to reach the value of 45-55 mm Hg. In spite of the advantages of this method, it is effective only for mild forms of bronchial asthma. The known method according to the certificate of authorship No. 1067640 is prior art in view of this invention.

One technical problem to be solved by this invention results from increasing the effectiveness of hypocarbia treating by elimination of CO₂ deficiency as a whole.

This problem is solved by a method of treating hypocarbia diseases by decreasing lung ventilation to the extent tolerant for a patient, monitoring the carbon dioxide (CO₂) increase rate in the exhaled air and maintaining its increase rate, decreasing lung ventilation to the extent tolerant for a patient is carried out at rest, and during exercise, increasing a CO₂ rate in the exhaled air which is maintained at not more than 2 mm Hg per day at rest, and 11 mm Hg when doing exercise, up to the level of 32.1 mm Hg when eliminating disease acute symptoms, and up to 55 mm Hg during long-term clinical remission.

The invention is illustrated by clinical examples. The content of CO₂ in the exhaled air is determined by well-known methods, including a known standard apparatus “Capnograph” and a test CO₂ measurement method based on the method according to a certificate of authorship No. 1593527.

### CLINICAL EXAMPLES

### Example 1

### Treatment of Alveolar Hypocarbia

P., 6 years old. Diagnosis: atopic bronchial asthma, severe form. RF π. Chronic vasomotor rhinitis. Polyvalent allergy.

Admission complaints: constant dyspnea, 6-7 asthmatic attacks, mainly during physical activity and at night; constant cough with hardly discharge sputum, sometimes up to vomiting; labored nasal breathing; weakness; troubled sleep; irritability; skin itch; constipations.

Objectively: moderately severe condition. Practically no nasal breathing, distant rales, severe expiratory dyspnea. Pale skin integument. Auscultatory identified: dispersed high and low-pitched dry rales, medium moist rales above all pulmonary tissue. Percussion data: vesiculotympanitic resonance. RR=30 per min. Heart, sounds are muffled, heart rate=118 per min, regular rhythm, ABP=100/60 mm Hg. Geographic tongue, abdominal wall tenderness in the epigastric area and in the right hypochondrium. CP=3 sec, pCO₂=22.4 mm Hg.

P. learned the method quickly, in 10-15 minutes acute severe asthma was stopped: nasal breathing was improved, cough and dyspnea decreased, heart rate was reduced up to 100 per minute. Auscultatory, the number of high-pitched dry rales was reduced, RR=20 per minute. On the 6th day of treating CP was increased up to 9-10 sec, level of pCO₂ was from 27.3 up to 31.8 mm Hg. There was no asthmatic attack and no need in medicine treatment, the boy became more active, exercise tolerance was increased.

On the 14th day CP was increased up to 20 second, pCO₂ up to 35.6 mm Hg, which was accompanied by further dyspnea decrease, sleep normalization, appetite improvement, decrease in weakness and sweating.

When CP came up to 30-40 sec, clinical presentations of allergy disappeared, including reaction to smells. At the examination: satisfactory condition. Skin integument and visible mucous are pure and have normal color. Breathing in lungs is vesicular, with harsh component in the lower parts. RR=14 per minute. Heart—pure sounds, heart rate—80 per min, ABP=90/60 mm Hg. Tongue is clear and wet. Abdomen is soft and painless.

During the follow-up period after 6 months there was no acute attack of bronchial asthma and allergic reaction, stable remission was achieved.

Indices

| | CP = 3 sec | CP = 40 sec |
|---|---|---|
| VC 1 | 0.3 | 1.5 |
| pCO₂ mm Hg | 27.3 | 35.6 |
| Clinical blood analysis: | | |
| leucocytes | 9.2 × 10/1 | 7.2 × 10/1 |
| eosinophiles | 23 | 3 |
| stab | 1 | 5 |
| segmental | 40 | 54 |
| lymphocytes | 31 | 37 |
| monocytes | 5 | 1 |
| ESR | 9 mm/h | 9 mm/h |

Chest X-ray picture when CP=40 sec. (one month after the beginning of the treatment): lung fields are clear in the lower parts, roots of lungs are indurated. No infiltrative and focal changes. Heart—no peculiarities.

### Example 2

### Treatment of Alveolar Hemohypocarbia

N., 8 years old. Diagnosis: mixed bronchial asthma, mild form. RF 1. Adenoidectomy in 2002. Chronic year-round vasomotor rhinitis, acute condition. Atopic dermatitis. Chronic gastritis, remission.

Admission complaints: no nasal breathing, mucus secretion from nose, cough with mucoid sputum worse at night.

Objectively: Satisfactory condition. Skin integuments are pure. Cheeks skin is dry. No nasal breathing. Face puffiness, hyperemia of conjunctivas. Harsh breathing in lungs, dispersed high-pitched dry rales in small numbers over all pulmonary tissue. RR=18 per minute. Heart—pure sounds, heart rate—100 per minute, regular rhythm, ABP=100/60 mm Hg. Abdomen is soft and painless. MP=12 sec, pCO₂=29 mm Hg.

From the first days of exercises nasal breathing is restored against reduction in breathing depth during physical activity.

After 7 days: free nasal breathing, no secretion from nose. Hacking and labored nasal breathing at night. MP was increased up to 16 sec, pCO₂ up to 300 mm Hg.

After 14 days: no complaints.

Objectively: Satisfactory condition. Skin integuments are pure. Free nasal breathing. Vesicular breathing in lungs, no rales. RR=18 per minute. Heart—pure sounds, heart rate—90 per minute regular rhythm, ABP—90/60 mm Hg. Abdomen is soft and painless. MP=20 sec, pCO₂=32.1 mm Hg.

Indices

| Clinical blood analysis: | MP = 12 sec | MP = 20 sec |
|---|---|---|
| leucocytes | 8.9 × 10/1 | 4.5 × 10/1 |
| eosinophiles | 7 | 4 |
| stab | 6 | 1 |
| segmental | 54 | 63 |
| lymphocytes | 30 | 27 |
| monocytes | 3 | 5 |
| ESR | 26 | 13 |
| pCO₂ mm Hg | 29 | 32.1 |

### Example 3

### Treatment of Alveolar Hypocarbia

K., 9 years. Diagnosis: mixed bronchial asthma, medium form, acute condition. Polyvalent allergy to food and household allergens. Chronic gastritis.

Admission complaints: dry attack-like cough, worse at night, dyspnea during physical activity, asthma attacks at night and up to 2-3 times in the daytime.

Objectively: moderately severe condition. Pale and pure skin integument. Expressed cyanosis of nasolabial triangle. Harsh breathing in lungs, dispersed multiple high-pitched dry rales over all pulmonary tissue. RR=24 per minute. Heart—sounds are muffled, heart rate=120 per minute. MP=3 sec., PCO₂=22.4 mm Hg.

During the first hour of exercises the pause was increased up to 9 seconds, pCO₂ up to 28 mm Hg. Attack was stopped.

After 6 months: K. feels well, hacking remains in case of diet imbalance. MP=70 sec., pCO₂=47 mm Hg. Last 4 months K. does not take medicines.

| Indices | | |
|---|---|---|
| | MP = 3 sec | MP = 70 sec |

| Clinical blood analysis: | | | | |
|---|---|---|---|---|
| leucocytes | 5.2 | | 7.06 | |
| stab | — | | — | |
| segmental | 42.0 | | 34.7 | |
| lymphocytes | 41.2 | | 49.6 | |
| monocytes | 8.4 | | 7.5 | |
| eosinophiles | 8.0 | | 5.1 | |
| basophiles | 0.4 | | 0.4 | |
| The amount of | | | | |
| immunoglobulines: | | | | |
| IgG (ME/ml) | 102 | | 174 | |
| IgA (ME/ml) | 138 | | 160 | |
| IgM (ME/ml) | 95 | | 73 | |
| IgE (ME/ml) | 29 | | 29 | |
| IgD (ME/ml) | — | | — | |
| Lymphocytic component | | | | |
| of immune system: | | | | |
| T-lymphocytes content | 60.0% | | 63.5% | |
| T-helpers content | 35.3% | | 46.4% | |
| T-supressors content | 29.2% | | 19.2% | |
| Immunoregulatory | 1.2 | | 2.4 | |
| balance | | | | |
| Natural killers | 9.9% | | 14.2% | |
| B-lymphocytes | | | 4.6% | |
| Activated lymphocytes | | | 3.6% | |
| expressing receptors | | | | |
| Mononuclears | | | 91.2% | |
| HLA-DR+(monocytes, | | | 6.2% | |
| B-lymphocytes, | | | | |
| activated | | | | |
| T-lymphocytes) | | | | |
| Phagocytic activity of | | | | |
| neutrophils: | | | | |
| spontaneous | 0.16 | | 0.29 | |
| induced | 0.26 | | 0.35 | |
| stimulation index | 1.6 | | 1.4 | |
| neutrophiles adhesion | 57.1% | | 58.7% | |
| Antioxidant status: | | | | |
| Content of malonic | 3.2 | mcmol/ml | 4.5 | mcmol/ml |
| aldehyde | | | | |
| Content of lactoferrin | 1.0 | mg/ml | 1.0 | mg/ml |

| | | |
|---|---|---|
| Catalase activity | 982.0 | 1074 |
| Ceruloplasmin activity | 0.54 | 0.56 |
| Coefficient of oxidation- | 1.7 | 1.6 |
| reduction balance | | |
| Interferon status: | | |
| spontaneous interferon | <2 | <2 |
| alfa - interferon | 320 | 320 |
| gamma - interferon | 32 | 32 |
| serum interferon | <2 | 2 |
| pCO₂ mm Hg | 22.4 | 47 |

### Example 4

### Treatment of Hemohypocarbia

Ch., 67 years old. Diagnosis: CHD, exertional and rest angina of HI fc, postinfarction cardiosclerosis (large-focal myocardial infarction). Essential hypertension of the m stage, arterial hypertension of the IH stage. Circulatory insufficiency DA. Discircular encephalopathy of π stage of complex origin (arterial hypertension and cerebral atherosclerosis). The consequences of acute stroke in the right hemisphere.

Admission complaints: anginal attacks during exercise and at rest, vertigo, frequent disorientation. Ch. takes up to 15 tablets of Nitroglycerin, Sustak-forte—up to 4 tablets daily, Cordaron 0.2-3 times daily.

CP=3 sec, pCO₂-22.4 mm Hg.

After 21 days: CP=15 sec, pCO₂-29 mm Hg. Feels well, hear attacks occur only during excessive exercise, BAP is normalized. Nitro medicines takes only in cases of hear pains.

After 3 months: Takes no medicines. Heart pains occur not more than 1-3 times monthly, are stopped by the method. He walks quickly 10-15 km daily, can run 100-200 meters.

After 1 year: CP=35 sec, pCO₂-39 mm Hg.

On ECG positive dynamics: “T” (−) in leads V2-V4 is changed by “T” (+) in these leads.

Earlier for treating the above-mentioned diseases it was used a method of treating hemohypocarbia, see certificate of authorship No. 1067640, Buteiko K. P., wherein for treating bronchial asthma it is necessary to normalize the content of CO₂ in blood, but in cases of medium and severe forms of bronchial asthma the content of CO₂ in blood is already above the line. So the “old” method is used only for mild forms of bronchial asthma, when there is a deficiency of CO₂ in air cells and in blood.

Thus, both methods allow to treat a mild form of bronchial asthma and all the other above-mentioned diseases, and a “new” method (hypocarbia treatment) allows to treat mild, medium and severe forms of bronchial asthma and all the other diseases (i.e. the diseases caused by the deficiency of CO₂, in this case in alveolar air—alveolar hypocarbia).

According to this invention, a Buteyko method of treating hypocarbia allows one to achieve a long-term remission with regard to the above-mentioned diseases.

The abolition of hypocarbia results in the recovery from the following diseases: acute (pneumonia, bronchitis . . . ) diseases and chronic diseases of respiratory system—chronic obstructive bronchitis, bronchial asthma (on any stage and in any form), lungs emphysema, diffuse pulmonary fibrosis, focal pneumosclerosis, multiple bronchiectasis; cardiovascular system diseases: essential hypertension, ischemic disease (in any stage and in any form); allergic diseases: chronic vasomotor rhinitis, pollinosis, urticaria, atopic dermatitis, etc.; secondary immunodeficiency states; and gastrointestinal tract diseases, chronic gastroduodenitis, stomach and duodenal ulcer, cholecystitis, pancreatitis and etc.

1. A method of treating hypocarbia diseases and states comprising: decreasing lung ventilation to an extent tolerant for a patient; monitoring a carbon dioxide (CO₂) increase rate in an exhaled air and maintaining an increase rate, the method including carrying a lung ventilation decrease to the extent tolerant for the patient at rest and during exercise, maintaining the CO₂ increase rate in the exhaled air at not more than 2 mm Hg per day at rest and at 11 mm Hg during exercise up to a level of 32.1 mm Hg for eliminating disease symptoms and up to 55 mm Hg for a long-term clinical remission.
